(19) United States
(12) Patent Application Publication
(10) Pub. No.: US 2022/0104796 A1
(43) Pub. Date: Apr. 7, 2022

(54) BIOPSY NEEDLE ASSEMBLY AND METHOD

(71) Applicant: Ozca Engineering Ltd., Hod HaSharon (IL)

(72) Inventors: Oz Cabiri, Hod HaSharon (IL); Ivan Sepetka, Los Altos, CA (US)

(73) Assignee: Ozca Engineering Ltd., Hod HaSharon (IL)

(21) Appl. No.: 17/102,491

(22) Filed: Nov. 24, 2020

Related U.S. Application Data
(60) Provisional application No. 63/087,435, filed on Oct. 5, 2020.

(51) Int. Cl. A61B 10/02; A61B 10/04; A61B 17/32; A61B 17/34

(52) U.S. Cl. CPC A61B 10/0266; A61B 10/04; A61B 2017/00831; A61B 17/3403; A61B 17/320016

(57) ABSTRACT

A method of obtaining a tissue specimen includes moving a biopsy needle through a vasculature and through a wall of a blood vessel at a target site. At the target site, the biopsy needle forms a puncture through a wall of said blood vessel at a non-perpendicular angle so that an inner-wall opening of the puncture is further downstream from an outer-wall opening of the puncture. The biopsy needle resects a portion of tissue. The biopsy needle reenters the puncture with the portion of the tissue trapped therein. The biopsy needle is withdrawn from the vasculature for subsequent inspection of the tissue.

### FIELD OF THE INVENTION

The present invention generally relates to biopsy tools and methods, and particularly to a biopsy needle assembly and method.

### BACKGROUND OF THE INVENTION

A brain biopsy is the removal of a small piece of a brain tissue for the diagnosis of brain abnormalities. A brain biopsy may be used to diagnose Alzheimer's disease, tumors, infections, inflammations, and other brain disorders. By examining the tissue sample under a microscope, the biopsy sample provides the doctors with information necessary for diagnosis and treatment. Generally, biopsy surgeries are categorized based on the technique and the needle size used for tissue extraction.

In an open biopsy, an incision is made in the skull and a small piece of tissue near the surface of the brain is removed. The tissue is sent to a pathologist, who examines it under a microscope and determines the type of disease.

In a needle biopsy, a needle is used to access tumors or lesions that are deeper in the brain. A hole is generally drilled into the skull for the needle to pass through. A stereotactic frame is used to guide the needle into the brain and into the abnormal lesion or tumor.

It is clear that such invasive procedures carry with them many risks to the patient. A less invasive and less traumatic procedure is clearly needed.

### SUMMARY OF THE INVENTION

The present invention seeks to provide a biopsy needle assembly and method, as is described more in detail hereinbelow. The invention is particularly applicable for brain biopsies, but can be used in any other organ of the body, such as but not limited to myocardial biopsy, muscle biopsy, lung biopsy, liver biopsy, kidney biopsy, uterine and ovarian biopsy, esophageal biopsy, stomach biopsy, intestinal biopsy, tumor biopsy (anywhere in the body) and others, for fast biopsy analysis. Moreover, the invention can be used for delivering drugs, therapeutic materials, radioactive materials and any other substances to the brain or any other organ of the body; the needle reaches the desired target at the desired orientation, and the substance can be delivered through the lumen of the needle.

In the present invention, a biopsy needle is guided through the vasculature, such as through the brain, or any other vasculature, to the target site. Any type of guiding catheter or other device can be used to bring the needle to the desired target site. Imaging such as CT, MRI, biplane fluoroscopy, 3D angiography, 4D fluoroscopy or 4D CT, may be used to guide the path of the needle. The needle is at the distal end of a highly bendable and flexible tool which can negotiate tortuous turns in the vasculature and which can pass through very small blood vessels or other lumens. At the target site, the needle punctures the blood vessel wall at a non-perpendicular angle so that the inner-wall opening of the puncture is further downstream from the outer-wall opening of the puncture. In this manner, the blood flow remains in the blood vessel and is not directed to flow out of the blood vessel. The biopsy needle resects a portion of the target tissue and reenters the angled puncture with the biopsy sample trapped therein. The needle with the biopsy sample is then withdrawn from the body for subsequent inspection and processing. Fluid (such as cerebrospinal fluid in the case of a brain biopsy) or other tissue matter surrounding the outside of the puncture is at a higher pressure than the fluid pressure inside the punctured blood vessel. This higher pressure tends to seal the puncture so that the puncture self-heals without need for additional steps, such as sutures (which would be difficult if not impossible to tie), stents or blocking structure.

### BRIEF DESCRIPTION OF THE DRAWINGS

The present invention will be understood and appreciated more fully from the following detailed description taken in conjunction with the drawings in which:

FIG. 1 is a simplified illustration of a biopsy needle assembly, which includes a flexible biopsy needle coupled to a micro-catheter, in accordance with a non-limiting embodiment of the present invention;

FIG. 2 is a simplified illustration of the biopsy needle assembly guided through vasculature of a patient;

FIG. 3 is an enlarged illustration of the biopsy needle puncturing the blood vessel wall at a non-perpendicular angle so that the inner-wall opening of the puncture is further downstream from the outer-wall opening of the puncture, in accordance with a non-limiting embodiment of the present invention;

FIG. 4 is a simplified illustration of a biopsy needle, which cuts in a backward (proximal) motion, in accordance with a non-limiting embodiment of the present invention (the needle being shown in the position after cutting);

FIG. 5 is a simplified illustration of the outer tube of the biopsy needle of FIG. 4;

FIG. 6 is a simplified illustration of the outer tube of the biopsy needle of FIG. 4 viewed from a different angle;

FIG. 7 is a simplified illustration of a biopsy needle, which cuts in a rotational motion, in accordance with a non-limiting embodiment of the present invention;

FIG. 8 is a simplified illustration of the outer tube of the biopsy needle of FIG. 7;

FIG. 9 is a simplified illustration of the inner cutting tube of the biopsy needle of FIG. 7;

FIG. 10 is a simplified illustration of a biopsy needle, which cuts in a backward (proximal) motion with a slanted cutting edge (not perpendicular to the longitudinal needle axis) behind (proximal to) the needle, in accordance with a non-limiting embodiment of the present invention (the needle being shown in the position after cutting);

FIG. 11 is a simplified illustration of the biopsy needle of FIG. 10, in the position before cutting;

FIG. 12 is a simplified illustration of the biopsy needle of FIG. 11 viewed from a different angle;

FIG. 13 is a simplified illustration of a biopsy needle, which cuts in a backward (proximal) motion with a straight cutting edge (perpendicular to the longitudinal needle axis) behind (proximal to) the needle, in accordance with a non-limiting embodiment of the present invention (the needle being shown in the position after cutting);

FIG. 14 is a simplified illustration of the biopsy needle of FIG. 13, in the position before cutting;

FIG. 15 is a simplified illustration of the biopsy needle of FIG. 14 viewed from a different angle;

FIG. 16 is a simplified illustration of the inner and outer parts of the biopsy needle of FIG. 13;

FIG. 17 is a simplified illustration of a biopsy needle with an inner cutting element that cuts in a backward (proximal) motion with respect to a slanted cutting edge of the outer tube of the needle, in accordance with a non-limiting embodiment of the present invention;

FIG. 18 is a simplified illustration of the biopsy needle of FIG. 17 at a different viewing angle;

FIG. 19 is a simplified illustration of a biopsy needle with a helical cutting element, in accordance with a non-limiting embodiment of the present invention;

FIG. 20 is a simplified pictorial illustration of another helical-cutting biopsy needle, wherein the needle is used to penetrate without cutting while the helical part is sharpened on its rear portion for accurate cutting.

FIG. 21 is a simplified illustration of the biopsy needle puncturing the blood vessel wall at a non-perpendicular angle so that the inner-wall opening of the puncture is further downstream from the outer-wall opening of the puncture, with temporary stent assistance during the diagonal puncture and specimen collection with controlled radial force of the stent expansion, in accordance with a non-limiting embodiment of the present invention;

FIG. 22 is a simplified illustration of stent assistance of wound closure after withdrawal of the biopsy needle, assisting hemostasis without blocking the blood flow; and

FIGS. 23-28 are simplified illustrations of an access catheter and torquer for use with the biopsy needle and its helical cutting element tube of the embodiment of FIG. 20.

### DETAILED DESCRIPTION OF EMBODIMENTS

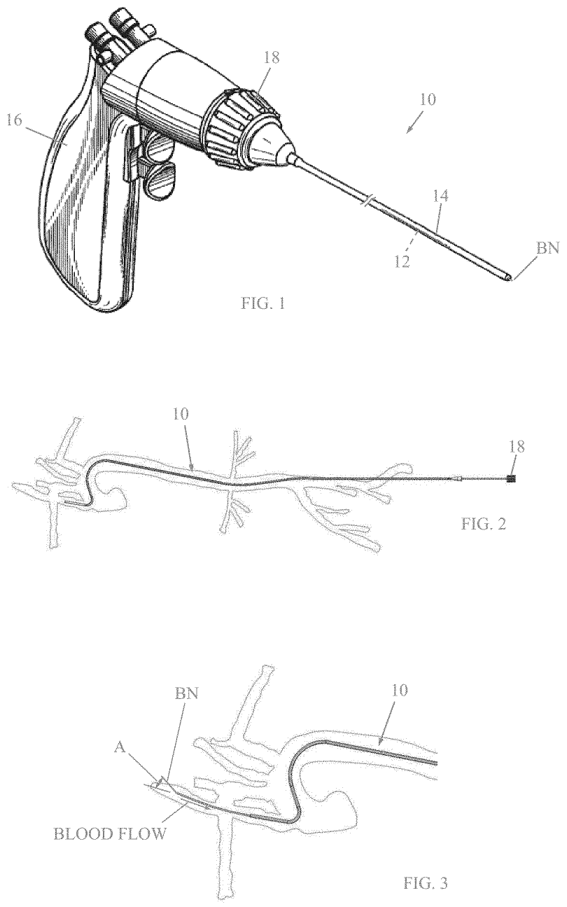

Reference is now made to FIG. 1, which illustrates a biopsy needle assembly 10, in accordance with a non-limiting embodiment of the present invention.

Without limitation, assembly 10 (also called a micro-catheter assembly) may include an internal tube 12 disposed inside an external tube 14. A distal end of internal tube 12 is fixedly joined to a distal end of external tube 14. The term “joined” encompasses any method for attaching the materials of the tubes together, such as but not limited to, welding, ultrasonic welding, thermal bonding, adhesive bonding, molding, mechanical fastening and others. The internal and external tubes 12 and 14 are arranged for longitudinal axial movement relative to one another (except for their distal ends which are joined together).

Assembly 10 may include a handle 16 that has a tube manipulator 18 (also referred to as control knob 18) for causing longitudinal axial movement of one of the internal and external tubes 12 and 14 relative to one another so as to cause the distal portions of the tubes to bend or curve or otherwise deform. The same or another tube manipulator may be used to lock the tubes 12 and 14 of assembly 10 completely or partially or not at all (i.e., unlocked so the tubes can move freely).

A biopsy needle BN is coupled to the distal end of either internal tube 12 or external tube 14. Non-limiting examples of biopsy needles are described hereinbelow. The biopsy needle may be coupled to the tube by any suitable means, such as but not limited to, joining (as defined above) or as one integral part of either internal tube 12 or external tube 14.

Reference is now made to FIG. 2. The assembly 10 can be introduced as in a standard angiographic procedure into a blood vessel, typically a vein, and advanced and guided to a desired blood vessel in the brain at the site of the tissue for which it is desired to collect a biopsy sample.

Reference is now made to FIG. 3. The biopsy needle BN punctures the blood vessel wall at a non-perpendicular angle A (such as, but not limited to, 15° or 10-30°) so that the inner-wall opening of the puncture is further downstream from the outer-wall opening of the puncture (this is also shown in FIG. 21). In this manner, the blood flow remains in the blood vessel and is not directed to flow out of the blood vessel. The biopsy needle resects a portion of the target tissue and reenters the angled puncture with the biopsy sample trapped therein. The needle with the biopsy sample is then withdrawn from the body for subsequent inspection and processing. Fluid (such as cerebrospinal fluid in the case of a brain biopsy) or other tissue matter surrounding the outside of the puncture is at a higher pressure (e.g., without limitation, 7-15 mm Hg) than the fluid pressure inside the punctured blood vessel (e.g., without limitation, 3-10 mm Hg). This higher pressure tends to seal the puncture so that the puncture self-heals without need for additional steps, such as sutures (which would be difficult if not impossible to tie), stents or blocking structure. When the procedure is over, the assembly 10 can be removed as in a standard angiographic procedure.

It is important to note that the invention also covers the situation the pressure inside the blood vessel is higher than the fluid pressure outside the blood vessel. The hemostasis will be achieved by the non-perpendicular puncture, and can be assisted by a stent, as described below in FIGS. 21-22.

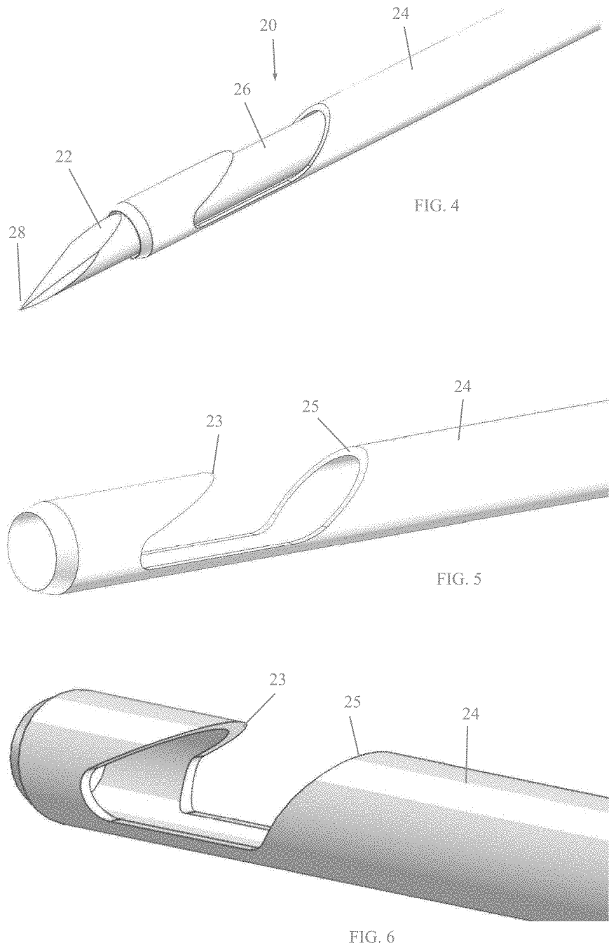

Reference is now made to FIGS. 4-6, which illustrate a biopsy needle 20, which cuts in a proximal motion, in accordance with a non-limiting embodiment of the present invention. Biopsy needle 20 includes an inner needle 22 that slides inside an outer tube 24. Inner needle 22 has a shaft 26 with a sharp tip 28 for piercing the blood vessel wall. The outer tube 24 is formed with distal and proximal slanted (not perpendicular to the longitudinal axis of the needle) cutting edges 23 and 25, respectively. Tissue may be cut by the slicing motion of needle 22 moving past cutting edge 23 or 25 and the cut tissue specimen is collected inside outer tube 24 (this is just one example of a tissue collecting volume). After completing the procedure, when the device is out of the body. the inner needle may be used to push the biopsy tissue from the outer tube.

The biopsy needle 20 (or needle 22) may be a radioactive needle or radioactive tipped needle, which can be used in conjunction with nuclear medicine imaging techniques to identify and localize abnormalities that may not be seen using other imaging techniques, or to emit radiation in the treatment of a lesion, for example. The radioactive portion can be detached and left in position where the lesion is located.

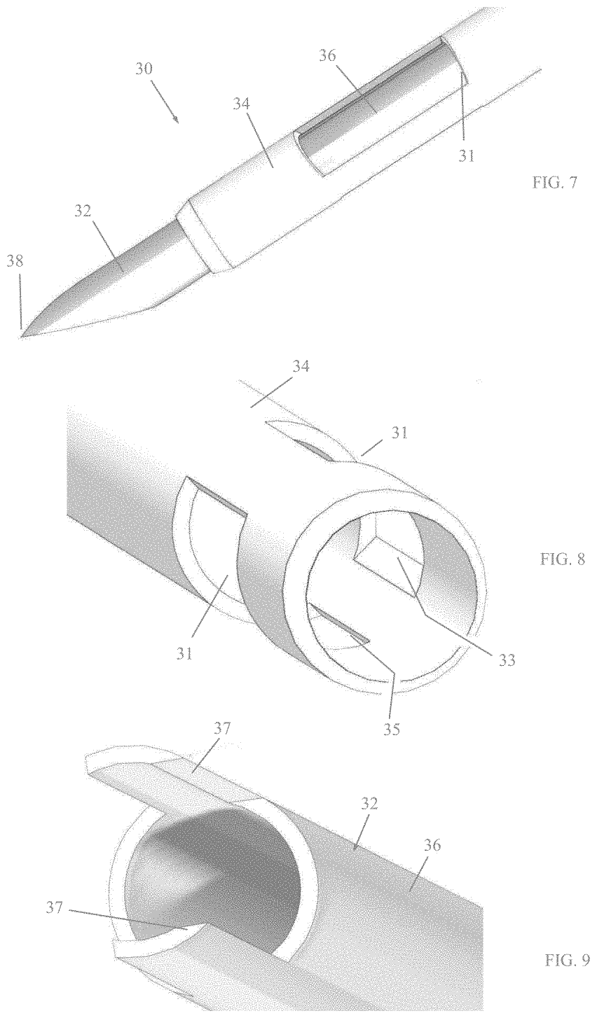

Reference is now made to FIGS. 7-9, which illustrate a biopsy needle 30, which cuts in a rotational motion, in accordance with a non-limiting embodiment of the present invention. Biopsy needle 30 includes an inner needle 32 that rotates inside an outer tube 34. Inner needle 32 has a shaft 36 with a sharp tip 38 for piercing the blood vessel wall. Inner needle 32 also has one or more cutting elements 37 (FIG. 9) that extend from shaft 36. The outer tube 34 is formed with one or more cutting edges 33 and 35, which may be positioned at different circumferential positions. The outer tube 34 is formed with one or more windows 31 that are bounded by cutting edges 33 and 35. Tissue may be cut by the rotary motion of cutting elements 37 of needle 32 moving past cutting edge 33 or 35. The cut tissue specimen is collected inside outer tube 34.

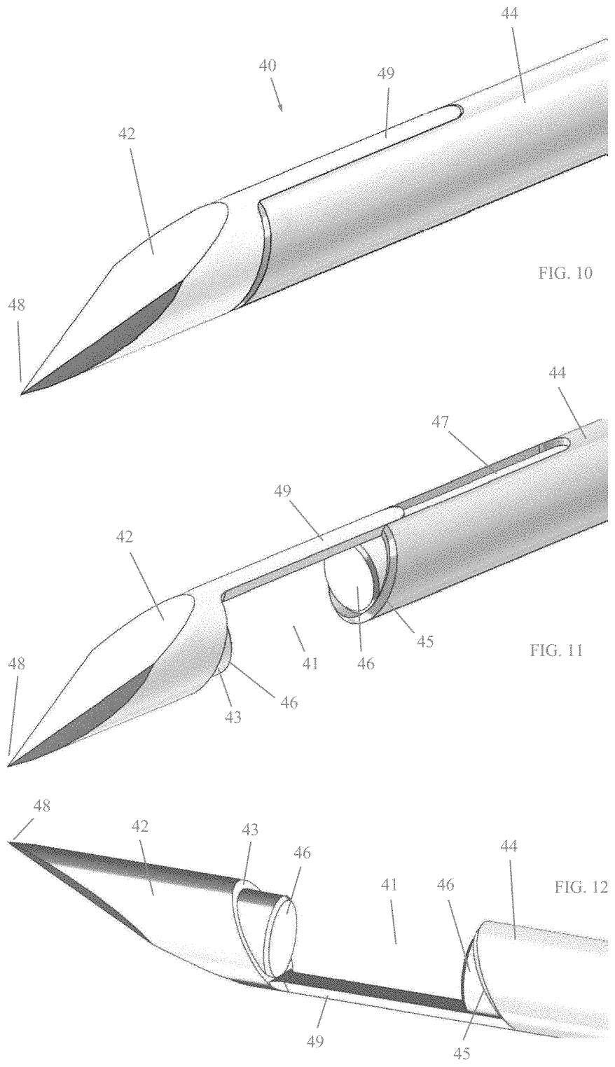

Reference is now made to FIGS. 10-12, which illustrate a biopsy needle 40, in accordance with a non-limiting embodiment of the present invention. Biopsy needle 40 includes an inner needle 42 that slides inside an outer tube 44. Inner needle 42 has a shaft 46 with a sharp tip 48 for piercing the blood vessel wall. The shaft 46 is formed with a span element 49 that connects distal and proximal portions of shaft 46 that are separated from each other by a gap 41. The outer tube 44 is formed with a slanted (not perpendicular to the longitudinal axis of the needle) cutting edge 45; needle 42 may be formed with a slanted cutting edge 43. Outer tube 44 is also formed with a channel 47 in which span element 49 can slide. Tissue may be cut by cutting edge 43 or 45 and the cut tissue specimen is collected inside outer tube 44.

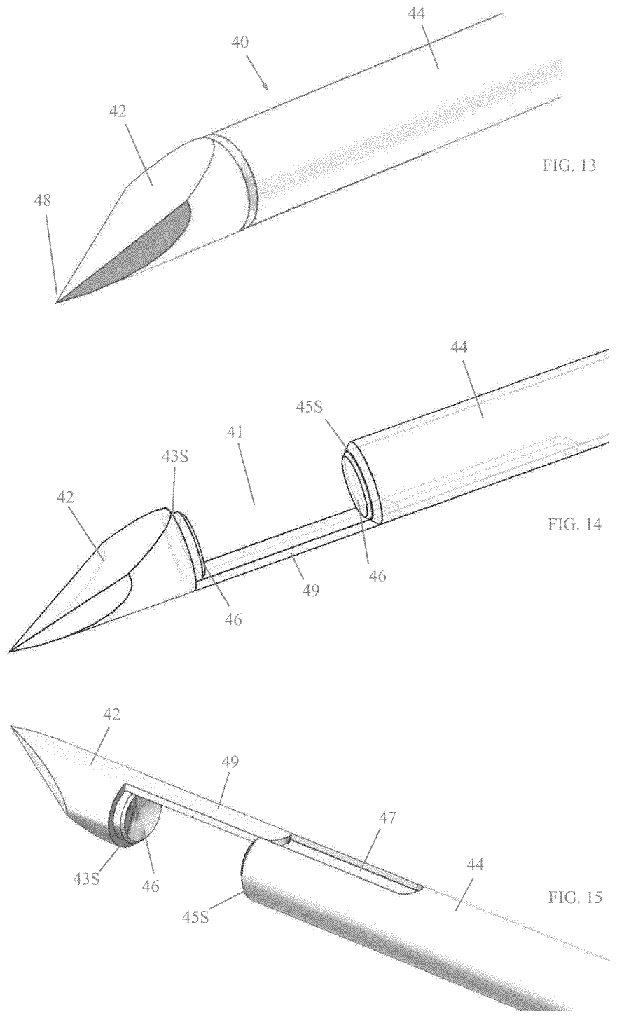

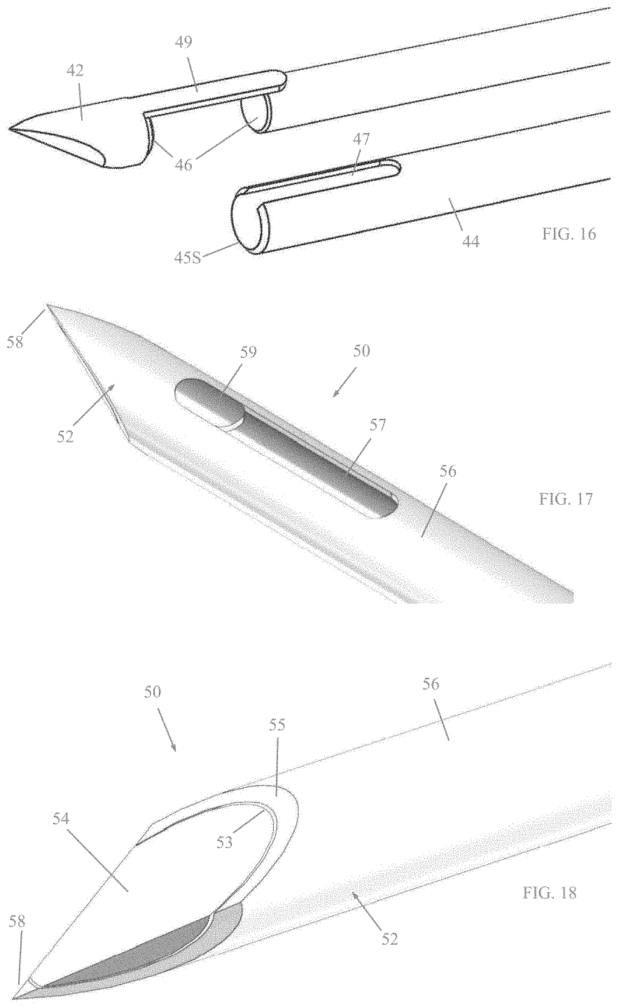

Reference is now made to FIGS. 13-16, which illustrate another version of biopsy needle 30, with like elements being designated by like numerals. The difference between the embodiment of FIGS. 13-16 and that of FIGS. 10-12, is that in the embodiment of FIGS. 13-16 instead of slanted cutting edges there are straight cutting edges (perpendicular to the longitudinal needle axis) 43S and 45S. In this configuration the tissue is pushed by the inner needle diagonally and may be released into the open cell (gap) 41 and then trimmed between the cutting edges 45S and 43S

Reference is now made to FIGS. 17-18, which illustrate a biopsy needle 50, in accordance with a non-limiting embodiment of the present invention. Biopsy needle 50 includes an outer needle 52 that has a shaft 56 with a sharp tip 58 for piercing the blood vessel wall. Needle 52 is formed with a slanted cutting edge 55. An inner cutting element 54 slides inside needle 52. The sliding motion is controlled by a lug 59 that slides in a channel 57 formed in needle shaft 56. The cutting element 54 is formed with a slanted cutting edge 53. Tissue may be cut by the slicing motion of cutting elements 53 and 55. The cut tissue specimen is collected inside needle 52.

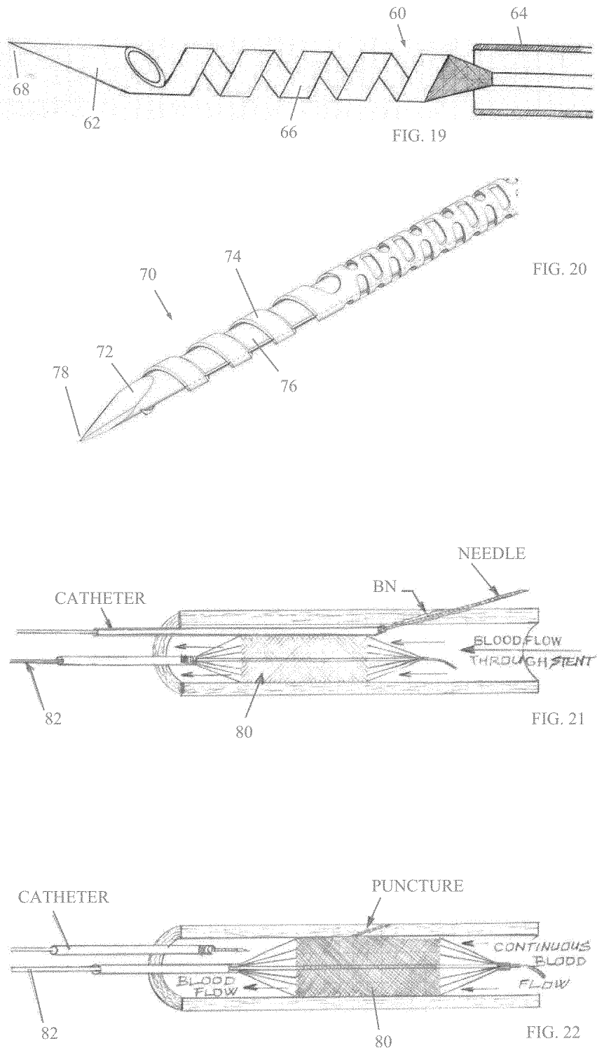

Reference is now made to FIG. 19, which illustrates a biopsy needle 60 with a helical cutting element, in accordance with a non-limiting embodiment of the present invention. Biopsy needle 60 includes an inner needle 62 which has a sharp distal tip 68 at a distal end of a helical shaft 66. Inner needle 62 slides inside an outer tube 64. The needle 62 penetrates into tissue by the corkscrew action of the helical shaft 66.

Reference is now made to FIG. 20, which illustrates a biopsy needle 70 with a helical cutting element, in accordance with another non-limiting embodiment of the present invention. Biopsy needle 70 includes an inner needle 72 which has a sharp distal tip 78 at a distal end of a shaft 76. Inner needle 72 slides inside an outer helical tube 74.

The tip 78 of needle 72 penetrates into tissue like any other needle. After needle penetration, the needle 72 may be pulled back (proximally) inside the outer helical tube 74. This leaves the sharp coil (like a pigtail anchor) to drill into the tumor with rotation to cut a biopsy sample.

The rear (proximal) part of the helical tube 74 is sharp. After helical tube 74 has been drilled into the tissue, the user can keep rotating tube 74 without distally advancing or proximally pulling back the tube 74; this rotation can cut the tissue sample and lock it in the tube for retrieval once the device is removed from the body.

To release the trapped tissue, after removal of the device from the body, the user can simply rotate tube 74 (such as clockwise) and push the inner needle 72 to release and eject the tissue sample.

The helical tube (“corkscrew”) 74 may have structural features for navigation in the vasculature and for capturing tissue samples. For example, the entire length of the corkscrew may be radiopaque and kink-resistant for safe navigation during its delivery and safe tissue penetration for the biopsy sample removal. Suitable materials for making tube 74, include without limitation, a drawn-filled tube made of nitinol, or a mixture of nitinol and platinum, or gold-plated or platinum-plated nitinol hypo-tube.

The wall thickness of the helical tube 74 can be reduced to provide some elongation when pulled back, which provides a type of coil or braid stretching (Chinese finger trap) effect. This effect can help secure the trapped tissue inside the corkscrew during removal. When pulled back, the helical tube 74 contracts against the tissue sample, thereby helping to trap the sample in tube 74. Helical tube 74 may extend from a laser-cut tube which has flexibility yet good torqueability.

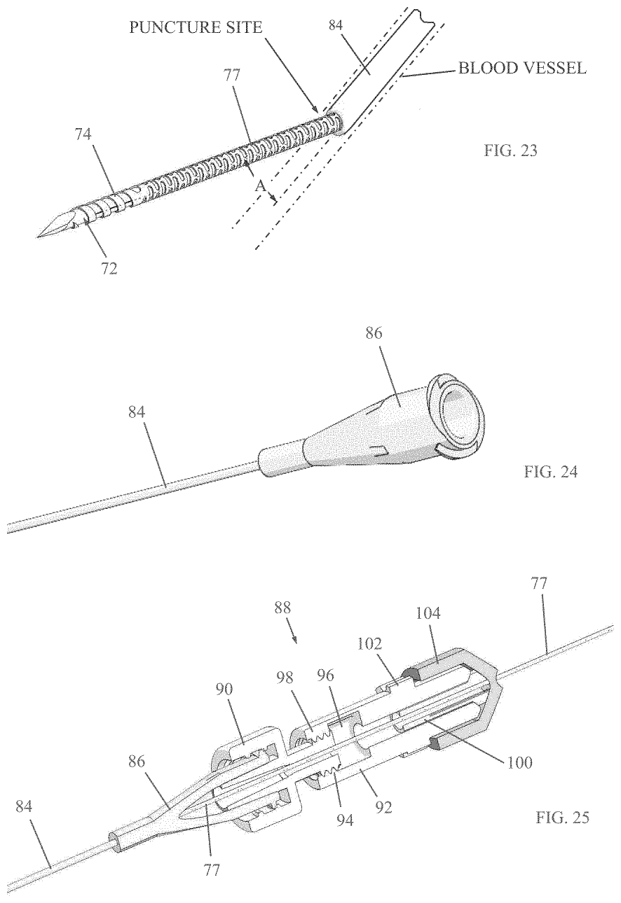

An access catheter and torquer for use with the biopsy needle 70 and its helical cutting element tube 74 is described below with reference to FIGS. 23-28.

Reference is now made to FIG. 21, which illustrates any of the biopsy needles BN of the invention puncturing the blood vessel wall at a non-perpendicular angle so that the inner-wall opening of the puncture is further downstream from the outer-wall opening of the puncture. A stent 80 may provide temporary assistance during the diagonal puncture and specimen collection with controlled radial force of the stent expansion. The stent 80 may be delivered over a guidewire 82, as is well known.

Reference is now made to FIG. 22. Here the stent 80 may assist in the wound closure of the puncture after withdrawal of the biopsy needle.

Reference is now made to FIGS. 23-28, which illustrate an access catheter and torquer for use with the biopsy needle 70 and its helical cutting element tube 74 of FIG. 20.

FIG. 23 illustrates the flexible outer helical tube 74 and its (laser-cut) proximal portion 77 assembled over the inner (penetration) needle 72 as before, and inserted in an access catheter 84.

FIG. 24 illustrates access catheter 84 with a proximal connector 86, such as a (male) Luer connector 86.

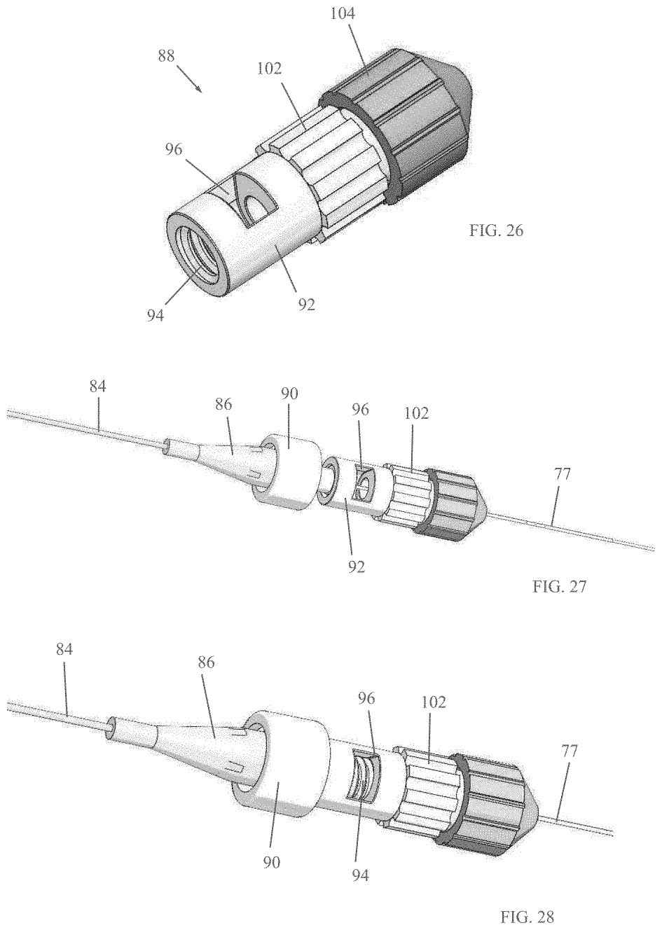

FIG. 25 illustrates the proximal Luer connector 86 coupled to a torquer 88 (which is also shown in FIG. 26). Torquer 88 may have a distal connector 90, such as a (female) Luer connector, so that the proximal connector 86 can mate with the distal connector 90, such as by the male Luer threads coupling with the female Luer threads.

As seen in FIGS. 25 and 26, torquer 88 may include a rotatable tube mover 92 formed with distal threads 94 and a viewing window 96. The distal (female) threads 94 may mate with (male) threads of a coupling member 98 (FIG. 25) that extends proximally from distal connector 90. The pitch of distal threads 94 (and of course the pitch of the threads of coupling member 98) is preferably exactly the same pitch as the helical coils of the helical tube 74 (FIGS. 20 and 23). This provides one-to-one correspondence of turning the torquer with advancement of the helical tube.

As seen in FIG. 25, the rotatable tube mover 92 may include a tube holder 100 (for securely holding the proximal portion 77 of the helical tube) and a turning knob 102 (shown partially in FIG. 25). Torquer 88 may have a proximal end cap 104. As seen in FIGS. 27-28, turning knob 102 may be used to rotate the rotatable tube mover 92.

In use, as mentioned before, the tip of needle 72 penetrates into tissue like any other needle. After needle penetration, the needle 72 may be pulled back (proximally) inside the outer helical tube 74. The torquer 88 is used to rotate helical tube 74, by turning knob 102 which advances the helical coils of tube 74 (FIG. 27) so they drill or corkscrew into the tumor and cut a biopsy sample. As mentioned before, the rear (proximal) part of the helical tube 74 is sharp. After helical tube 74 has been drilled into the tissue, the distal threads 94 have advanced past the threads of coupling member 98; the distal face of rotatable tube mover 92 now abuts against the proximal face of coupling member 98. At this point, the helical tube cannot advance anymore distally and the distal threads 94 are visible in viewing window 96 (FIG. 28). The user can now keep rotating the helical tube without distally advancing or proximally pulling back the helical tube; this rotation can cut the tissue sample and lock it in the tube for retrieval once the device is removed from the body.

To release the trapped tissue, after removal of the device from the body, the user can simply rotate the helical tube (such as clockwise) and push the inner needle to release and eject the tissue sample.

What is claimed is:

1. A method of obtaining a tissue specimen comprising:
moving a biopsy needle through a vasculature and through a wall of a blood vessel at a target site, guidance of said biopsy needle being provided by an imaging modality, said needle being coupled to a bendable and flexible tool;
at the target site, using said biopsy needle to form a puncture through a wall of said blood vessel at a non-perpendicular angle so that an inner-wall opening of said puncture is further downstream from an outer-wall opening of said puncture;
using said biopsy needle to resect a portion of tissue;
causing said biopsy needle to reenter said puncture with said portion of the tissue trapped therein; and
withdrawing said biopsy needle from the vasculature for subsequent inspection of said portion of the tissue.

2. The method according to claim 1, wherein fluid or other tissue matter surrounding an outside of said puncture is at a higher pressure than fluid pressure inside said blood vessel which has been punctured.

3. The method according to claim 1, wherein said tissue is in a brain of a patient.

4. The method according to claim 3, wherein cerebrospinal fluid or other tissue matter surrounding an outside of said puncture is at a higher pressure than fluid pressure inside said blood vessel which has been punctured.

5. The method according to claim 1, wherein said biopsy needle comprises a radioactive portion.

6. The method according to claim 1, wherein using said biopsy needle to resect the portion of tissue is done by linear motion of cutting edges of said biopsy needle.

7. The method according to claim 1, wherein using said biopsy needle to resect the portion of tissue is done by rotational motion of cutting edges of said biopsy needle.

8. The method according to claim 1, wherein using said biopsy needle to resect the portion of tissue is done by corkscrew motion of cutting edges of said biopsy needle.

9. The method according to claim 1, wherein said biopsy needle comprises a radioactive portion which is left in the tissue.

10. A biopsy needle assembly comprising:
a biopsy needle coupled to a micro-catheter assembly, said biopsy needle comprising a sharp tip and at least one cutting edge suitable for cutting a tissue specimen, said biopsy needle also comprising a volume for collected therein said tissue specimen, and wherein said micro-catheter assembly has an operative configuration in which said sharp tip is directed to form a puncture through a wall of a blood vessel at a non-perpendicular angle so that an inner-wall opening of said puncture is further downstream from an outer-wall opening of said puncture.

11. The biopsy needle assembly according to claim 10, wherein said at least one cutting edge is straight.

12. The biopsy needle assembly according to claim 10, wherein said at least one cutting edge is slanted.

13. The biopsy needle assembly according to claim 10, wherein said at least one cutting edge is helical.

14. The biopsy needle assembly according to claim 10, wherein said biopsy needle comprises an inner needle that slides inside an outer tube, and said outer tube is formed with at least one cutting edge.

15. The biopsy needle assembly according to claim 10, wherein said biopsy needle comprises an inner needle that slides inside an outer tube, and said outer tube is a helical tube with at least one cutting edge.

16. The biopsy needle assembly according to claim 10, wherein said biopsy needle comprises an inner needle that rotates inside an outer tube, and said inner needle and said outer tube each have one or more cutting edges.

17. The biopsy needle assembly according to claim 15, wherein said helical tube elongates when pulled proximally.

18. The biopsy needle assembly according to claim 10, wherein said helical tube is coupled to a torquer that comprises a rotatable tube mover.

19. The biopsy needle assembly according to claim 18, wherein said rotatable tube mover is formed with threads with a pitch identical to a pitch of helical coils of said helical tube.
